(19) United States
(12) Patent Application Publication
(10) Pub. No.: US 2019/0290614 A1
(43) Pub. Date: Sep. 26, 2019

(54) Methods for Reducing Recurrence of Tumors

(71) Applicant: Beth Israel Deaconess Medical Center, Inc., Boston, MA (US)

(72) Inventors: Vikas Sukhatme, Newton, MA (US); Dipak Panigrahy, Whitman, MA (US)

(21) Appl. No.: 16/348,782

(22) PCT Filed: Nov. 8, 2017
(86) PCT No.: PCT/US17/60599

§ 371 Date: May 9, 2019

Related U.S. Application Data
(60) Provisional application No. 62/419,785, filed on Nov. 9, 2016.

(51) Int. Cl. A61K 31/407; A61P 35/00; A61K 39/395; A61K 31/635; A61P 35/04

(52) U.S. Cl. CPC A61K 31/407; A61P 35/00; A61K 39/3955; A61K 31/635; A61P 35/04; A61K 39/39558

(57) ABSTRACT

Methods of reducing the risk of cancer after surgery, e.g., of cancer recurrence, by administering COX-1 selective inhibitors prior to surgery or biopsy. Alternatively or in addition, other anti-platelet agents and/or TGF-β inhibition and/or IL-6 inhibition and/or inhibitors of the PD-1/PD-L1 axis, as well as combinations thereof.

### CLAIM OF PRIORITY

This application claims the benefit of U.S. Provisional Patent Application Ser. No. 62/419,785, filed on Nov. 9, 2016. The entire contents of the foregoing are hereby incorporated by reference.

### TECHNICAL FIELD

The present application relates, at least in part, to methods of reducing the risk of cancer after surgery, e.g., of cancer recurrence, by administering COX-1 selective inhibitors prior to surgery or biopsy. Alternatively or in addition, other anti-platelet agents and/or TGF-β inhibition and/or IL-6 inhibition and/or inhibitors of the PD-1/PD-L1 axis, as well as combinations thereof.

### BACKGROUND

Recurrence of disease remains the greatest threat to the survival of cancer patients (1). The treatment of loco-regionally disease is often surgery+/−radiation therapy+/−chemotherapy. However, surgery is a double-edged sword—it can be life-saving in many instances, yet has long-term, pro-tumorigenic potential, resulting in accelerated dormant tumor metastasis by stimulating inflammation (4); surgery can stimulate tumor dormancy escape and increase tumor recurrence (2). Surgery promotes various pro-angiogenic and pro-inflammatory cytokines factors, as well as reduces anti-angiogenic inhibitors, thereby stimulating metastasis (23-25). Post-operative tumor recurrence and metastasis present a major challenge in cancer treatment.

### SUMMARY

Conventional perioperative general anesthesia agents including morphine, ketamine, thiopental, or halothane suppress immune functions and stimulate tumor metastasis in experimental animal models and patients (19-21). Surgery and anesthesia act as a “stress reaction” to reduce cellular immunity in the post-operative period. During the perioperative period, the benefit of a nonsteroidal anti-inflammatory drug (NSAID) NSAID such as ketorolac and diclofenac was suggested in retrospective human data in lung and breast cancer (see (22) and reference therein to earlier mastectomy data), although a similar retrospective study found no benefit of perioperative ketorolac after radical prostatectomy for prostate cancer (Forget et al., Eur J Anaesthesiol 2011; 28:830-5).

As shown herein, ketorolac administered pre-surgically (but not post-surgically) maintained tumor cell dormancy in multiple models and inhibited biopsy-stimulated metastasis. Indeed, a single pre-operative dose improved survival in lung cancer, breast cancer and melanoma models and in fact appeared to cure a subset of mice by a T cell mediated mechanism. Furthermore, this effect was due to COX-1 inhibition since three different selective COX-1 inhibitors appeared to mimic the ketorolac data, while COX-2 inhibitors did not. Furthermore, a single dose of ketorolac in combination with a platelet aggregation inhibitor (MWReg30), a TGFβ inhibitor (1D11.16.8), or platelet depletion in combination with the TGFβ inhibitor resulted in a reduction cancer recurrence after tumor resection. Platelet aggregation inhibition alone with MWReg30 Ab also gave survival benefit. In addition, anti-PD1 Ab and ketorolac resulted in long-term survivors in approximately 80% of cases. Also, inhibitors of IL-6 and TGFβ (e.g., antibodies) may enhance long term survival. This is worth noting, since this combination should not increase risk of bleeding.

Thus, as shown herein, administration of COX-1 selective inhibitors prior to surgery or biopsy can reduce the risk of tumor recurrence and tumor dormancy escape. Alternatively or in addition, other anti-platelet agents and/or TGF-β inhibition and/or IL-6 inhibition and/or inhibitors of the PD-1/PD-L1 axis, as well as combinations thereof, can be used to inhibit tumor recurrence.

Thus, provided herein are methods for reducing the risk of cancer occurrence or recurrence in a subject undergoing a surgical procedure. The methods include administering to the subject a therapeutically effective amount of one or more of:

(i) one or more anti-platelet agents, preferably a COX-1 specific inhibitor and/or anti-CD41 antibody,
(ii) a TGF-β inhibitor;
(iii) an IL-6 inhibitor; and/or
(iv) a checkpoint inhibitor, preferably a Programmed cell death protein 1 (PD-1) or programmed death ligand-1 (PD-L1) inhibitor,
wherein the administering is performed before the surgical procedure.

Also provided herein are methods for preparing a subject for a surgical procedure, or for inducing an adaptive immune response to a cancer in a subject (e.g., a subject who has cancer, whether diagnosed or undiagnosed). The methods include administering to the subject a therapeutically effective amount of one or more of:

(i) an anti-platelet agent, preferably a COX-1 specific inhibitor,
(ii) TGF-β inhibitor;
(iii) an IL-6 inhibitor; and/or
(iv) a checkpoint inhibitor, preferably a Programmed cell death protein 1 (PD-1) or programmed death ligand-1 (PD-L1) inhibitor,
and subsequently performing a surgical procedure in the subject.

Also provided herein are compositions comprising two or more of (i) one or more anti-platelet agents, preferably including at least one COX-1 specific inhibitor,

(ii) TGF-β inhibitor;
(iii) an IL-6 inhibitor; and/or
(iv) a checkpoint inhibitor, preferably a Programmed cell death protein 1 (PD-1) or programmed death ligand-1 (PD-L1) inhibitor.

Further provided herein are one or more of i) one or more anti-platelet agents, preferably including at least one COX-1 specific inhibitor,

(ii) TGF-β inhibitor;

(iii) an IL-6 inhibitor; and/or

(iv) a checkpoint inhibitor, preferably a Programmed cell death protein 1 (PD-1) or programmed death ligand-1 (PD-L1) inhibitor for use in reducing the risk of cancer occurrence or recurrence in a subject undergoing a surgical procedure, and/or for use in inducing an adaptive immune response to a cancer in a subject (e.g., a subject who has cancer, whether diagnosed or undiagnosed) who is about to undergo a surgical procedure.

In some embodiments, the anti-platelet agent is a COX-1 Specific Inhibitor. In some embodiments, the COX-1 Specific Inhibitor is triflusal (2-acetyloxy-4-(trifluoromethyl)benzoic acid), aspirin, SC-560 (5-(4-Chlorophenyl)-1-(4-methoxyphenyl)-3-trifluoromethyl pyrazole), FR122047, mofezolac, P6, tenidap, ketorolac, Valeryl salicylate, or TFAP. In some embodiments, the COX-1 inhibitor does not affect COX-2 activity at the administered dose, or at any dose.

In some embodiments, the anti-platelet agent is an adenosine diphosphate (ADP) receptor inhibitor, a phosphodiesterase inhibitor; a protease-activated receptor-1 (PAR-1) antagonist; a Glycoprotein IIB/IIIA (GPIIB/IIA) inhibitor; an anti-CD41 antibody; an adenosine reuptake inhibitor; or a thromboxane inhibitor.

In some embodiments, the TGF-β inhibitor is an anti-TGF-beta antibody, antisense oligodeoxynucleotide, small molecule inhibitor of TGF-beta, or TGF-beta receptor inhibitor. In some embodiments, the anti-TGF-beta antibody is fresolimumab, Infliximab, Metelimumab, Lerdelimumab, GC-1008); the antisense oligodeoxynucleotide is Trabedersen; the small molecule inhibitor of TGF-beta is LY2157299, LY2382770, Lucanix, or Disitertide; or the TGF-beta receptor inhibitor is Galunisertib, TEW-7197, PF-03446962, or IMC-TR1.

In some embodiments, the IL-6 Inhibitor is an anti-IL-6 antibody or small molecule or peptide inhibitor. In some embodiments, the anti-IL-6 antibody is siltuximab, tocilizumab (atlizumab), sarilumab, olokizumab (CDP6038), elsilimomab, Clazakizumab, sirukumab (CNTO 136), or ALX-0061; or the small molecule or peptide inhibitor is CPSI-2364, Semapimod, ARGX-109, FE301, FM101, or soluble gp130 or sgf130Fc.

In some embodiments, the checkpoint inhibitor is an inhibitor of PD-1/PD-L1 is an anti-PD1 or anti-PD-L1 antibody. In some embodiments, the anti-PD1 is Nivolumab, pembrolizumab/MK-3475, Pidilizumab (CT-011), AMP-224, or AMP-554; or the anti-PDL1 is BMS-936559, MEDI4736, Atezolizumab (MPDL3280A), or Avelumab (MSB0010718C).

In some embodiments, the administering is performed within 4 hours before the surgical procedure begins.

In some embodiments, the administering is performed within 30 hours before the surgical procedure begins.

In some embodiments, the surgical procedure is a biopsy or surgical resection of a tumor.

In some embodiments, the administering is systemic

In some embodiments, the administering is local to the surgical site.

In some embodiments, the methods include administering epinephrine to the surgical site.

In some embodiments, the subject has been diagnosed with a cancer.

In some embodiments, the surgical procedure is cancer resection.

In some embodiments, the subject has not been diagnosed with cancer.

In some embodiments, the surgical procedure is unrelated to a cancer in the subject, e.g., is not a cancer biopsy or resection.

Also provided are compositions as described herein for use in the methods described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. Pre-operative Selective COX-1 Inhibition Inhibits Surgery-Induced Primary Tumor Growth and Metastasis

(A) Pre-operative, but not post-operative, ketorolac and celecoxib, prolonged survival in a spontaneous LLC lung metastasis model after primary tumor resection. Pre-operative, but not post-operative ketorolac, resulted in long term survival in approximately 50% of mice in a syngeneic LLC lung cancer model. Immunohistochemistry confirmed micrometastasis on day of tumor resection with no evidence of tumor micrometastasis in mice treated with ketorolac treated for 180 to 200 days. Ketorolac, 7.5 mg/kg intraperitoneally (I.P.); Celecoxib 30 mg/kg bid po; *p=0.0039 (Day 24) pre-operative (2 hrs prior to surgery) vs post-operative (2 hrs post-surgery).

(B) COX-1 selective, but not COX-2 selective inhibitor celecoxib and non-selective COX inhibitor indomethacin, mimicked the action of ketorolac in prolonging survival after primary LLC tumor resection. Ketorolac (7.5 mg/kg i.p.); celecoxib (60 mg/kg/oral gavage); indomethacin (3 mg/kg/i.p.); FR122047 (20 mg/kg i.p.); TFAP (30 mg/kg i.p.); SC-560 (30 mg/kg i.p.).

(C) Ketorolac prolonged survival in a surgery-stimulated spontaneous tumor model (genetically engineered murine breast tumor model (MMTV-PyMT).

(D) Pre-operative ketorolac prolonged survival in a spontaneous E0771 breast cancer metastasis model after primary tumor resection.

FIGS. 2A-D. Pre-operative Ketorolac Suppressed Surgery-Stimulated Primary Tumor Growth and Biopsy-Stimulated Metastasis

(A-C) Surgery (i.e. laparotomy) stimulated primary tumor dormancy escape in a B16F10 melanoma (1×10³ cells), Lewis lung carcinoma (LLC 1×10⁴ cells), and EL4 lymphoma (1×10⁴ cells). Images show representative tumors on Day 35 post-injection; Scale bar, 1 cm. Ketorolac administration pre-surgically inhibited surgery-induced tumor dormancy escape.

(D) Liver biopsy stimulated lung metastasis post primary tumor resection (T241 fibrosarcoma). Ketorolac administration pre-surgically inhibited biopsy-stimulated lung metastasis.

FIGS. 3A-C. Adaptive Immunity is Critical to Anti-Metastatic Activity of Ketorolac.

(A) Ketorolac did not have anti-metastatic activity in nude athymic C57BL/6 mice when administered prior to LLC tumor resection.

(B) Ketorolac did not inhibit metastases in SCID mice after human lung tumor (H460) primary tumor resection.

(C) Ketorolac did not inhibit laparotomy-induced tumor dormancy escape in Rag1 KO Mice (B16F10, 1×10³).

FIGS. 4A-C. Platelet Depletion/anti-aggregation agent and TGFβ Inhibition synergize with Ketorolac to prevent tumor recurrence.

(A) Immunocompetent mice (C57BL6) 200 days post LLC tumor resection administered ketorolac preoperatively resisted a second tumor challenge (LLC). In contrast, these same mice exhibited rapid tumor growth when injected with a third tumor challenge with a different tumor-type (B16F10 melanoma).

(B) Ketorolac prevented tumor metastasis when administered prior to surgery for LLC tumor resection in WT and COX-2 KO mice. In contrast, celecoxib did not prevent metastasis in WT and COX-2 KO mice.

(C) Ketorolac in combination with platelet depletion (MWReg30; 2 ug/mouse), TGF-β inhibitor (1 mg/mouse), and TGF-B inhibitor in combination with platelet depletion (MWReg30) prevented tumor metastasis post primary LLC tumor resection. IL-6 neutralizing antibody, 1 mg/mouse.

FIGS. 5A-B. Combination of Preoperative Ketorolac and Checkpoint Inhibitor Exhibited Synergistic Anti-Metastatic Activity.

(A-B) Preoperative Ketorolac and PD-1 prolonged survival post-primary tumor resection (LLC or EL4) compared to ketorolac or PD-1 alone.

FIG. 6. Preoperative COX-1 Inhibition Prevented Growth Or Proliferation Of Dormant Tumor Cells.

Hematoxylin & Eosin analysis of lungs at the time of surgery (LLC tumor resection) (Day 0) revealed abundant LLC tumor cells. In dramatic contrast, in lungs from mice treated 2 hours prior to surgery with ketorolac no tumor cells were detectable after 240 days post-surgery (LLC tumor resection). Representative micrographs of 4 different mice per group (total of 10 mice/group).

### DETAILED DESCRIPTION

One-third of cancer patients will develop local or systemic recurrence of disease following surgical resection. Post-operative metastases develop from pre-existing micrometastases and remaining tumor cells left after the excision of the primary tumor. The initiation of new metastases from these micrometastases occurs presumably through the release of prostaglandins and stress hormones (e.g. catecholamines and glucocorticoids) (3, 4). The mechanism of tumor recurrence is also clinically relevant for patients who do not present primary symptoms of cancer. It has been documented through autopsies of trauma victims, that otherwise healthy individuals harbor microscopic primary cancers: up to 99% thyroid cancer, 30-40% prostate cancer, and 30-40% breast cancer (7, 8). Dormant cancer cells may remain asymptomatic, non-detectable, and occult (6); however, they maintain proliferative, tumorigenic potential. In fact, surgery alone can increase cancer risk among individuals without a history of clinical cancer (5). Biopsies of tumors can also stimulate distant metastasis (9). Thus, anesthesia, surgery, or a biopsy could activate tumor dormancy escape in an otherwise healthy person undergoing non-cancer surgery.

In clinical studies, non-steroidal anti-inflammatory drugs (NSAIDs) have been associated with cancer preventative activity presumably via inhibition of cyclooxygenase (COX)-2 (10, 11). However, COX-2 inhibitors, such as celecoxib, have not been efficacious in clinical cancer trials. There clinical use has also been severely limited due to associated cardiovascular and gastrointestinal toxicity (12, 13). Cyclooxygenase (COX) inhibitors are also used perioperatively to reduce post-operative pain. We hypothesized that pre-operative selective COX-1 inhibition may prevent tumor recurrence by inhibiting surgery-induced inflammation. As demonstrated herein, selective COX-1 inhibition prior to surgery is a novel target to prevent tumor recurrence after surgery or biopsy. The present examples utilized ketorolac tromethamine (generic name is Toradol), an FDA-approved NSAID COX-inhibitor (non-selective, but with a preference for COX-1; see (14)) to demonstrate this in animal models of cancer resection. Ketorolac is a non-toxic, inexpensive and widely used analgesic; often utilized for short-term (less than 5 day) post-operative analgesia (15). Ketorolac acts mainly through reversible inhibition of the COX-1 pathway, which biosynthesizes prostaglandins and thromboxanes from arachidonic acid, thereby inhibiting (inter alia) platelet aggregation (16). Three other selective COX-1 inhibitors SC-560 (5-(4-Chlorophenyl)-1-(4-methoxyphenyl)-3-trifluoromethyl pyrazole); N-(5-amino-2-pyridinyl)-4-trifluoromethylbenzamide (TFAP); and FR 122047 (1-[[4,5-bis(4-Methoxyphenyl)-2-thiazolyl]carbonyl]-4-methylpiperazine hydrochloride)) showed the same tumor-suppressive effect.

As shown herein, other agents that inhibit platelet aggregation and degranulation, or the downstream effects of platelet aggregation and degranulation, given pre-operatively can also have the same effect (e.g., agents such as inhibitors or antagonists of Interleukin 6 (IL-6) and/or transforming growth factor beta (TGF-beta); other platelet inhibitors such as anti-CD41 antibodies, and checkpoint inhibitors such as anti-PD 1 antibodies that maintain or accentuate the T cell response) can be used, either alone or in combination, e.g., with each other or with ketorolac. For example, the present data shows that a COX-1 inhibitor (e.g., ketorolac) alone or used in combination with IL-6 or TGF-beta antagonists, other platelet inhibitors, and checkpoint inhibitors such as Anti-PD1 antibodies reduces the risk of tumor recurrence after surgery or biopsy, and can even raise a tumor-specific and durable adaptive immune response in subjects undergoing surgical interventions.

In dramatic contrast, most anesthetic drugs used perioperatively, including ketamine, thiopental, propofol, nitrous oxide, morphine and fentanyl, are associated with accelerated metastatic development (17, 18). The standard of care in peri-operative analgesia includes the use of opioids (e.g., morphine) and non-steroidal anti-inflammatory drugs (NSAIDs). As inexpensive drugs with minimal toxicity, non-steroidal anti-inflammatory drugs (NSAIDs) are available for postoperative pain management (26). Ketorolac is inexpensive and routinely used during surgery, contributing to multimodal anesthesia. Ketorolac was effective in preventing tumor recurrence after surgery or biopsy in our various models. Celecoxib alone did not improve survival of tumor-bearing mice (27). Another COX-2 inhibitor, etodolac, also when used alone did not prevent spontaneous postoperative metastasis in a mouse model (28). This study used a single subcutaneous injection of the drug before the resection. The standard analgesia morphine stimulates angiogenesis, tumor cell proliferation, primary tumor growth and metastasis (27, 29). Moreover, opiates display numerous toxicities, including nausea, vomiting, and respiratory depression. Thus, the use of morphine and other opiates have recently been minimized perioperatively, especially in cancer patients. An approach to reduce the dose of an opioid while keeping adequate analgesia is to combine the opioid with a NSAID (30).

The perioperative period is a critical time of metastatic susceptibility (31). Metastasis in cancer patients after surgery is stimulated by immunosuppression. NSAIDs are the least immunosuppressive after surgery compared to morphine and other peri-operative analgesics (32). Currently, during surgery no active systemic antineoplastic agents are utilized for fear of worsening the surgical outcome. Standard chemotherapy regimens, even when given in the neoadjuvant setting, are administered to patients well in advance of cancer debulking surgery (31). Thus, there is typically a period of at least a month before surgery and usually 1 to 2 months afterward when the patient is not being treated systemically with antineoplastic agents such as chemotherapy to allow for recovery.

The present data shows that in this perioperative period, especially the administration of COX-1 selective inhibitors preoperatively, may prevent tumor recurrence after surgery or biopsy. Ketorolac has been demonstrated to be safe and effective analgesia during cancer surgery, for example nephrectomy for renal cortical tumors (16). The only major side effect of ketorolac is an increased bleeding time and this increased risk of bleeding complications results from decreased biosynthesis of thromboxane A2, which is important in promoting platelet aggregation. However, multiple studies have demonstrated that patients administered ketorolac demonstrate improved overall pain management, without an increased risk of bleeding complications or other toxicities (16). A further advantage of ketorolac is the overall low cost (16). Studies also support that ketorolac in combination with opiates is superior to opioid analgesia alone in the management of post-operative pain for patients (16).

The anti-inflammatory and anti-metastatic activity of ketorolac, combined with minimal toxicity, suggest that ketorolac and other selective COX-1 inhibitors would be ideal to use in the preoperative and pre-biopsy setting. Also, as noted above, other anti-platelet agents and/or TGF-β inhibition and/or IL-6 inhibition and/or inhibitors of the PD-1/PD-L1 axis can also be used to inhibit tumor recurrence.

Methods of Treatment

The methods described herein include methods for the treatment of subjects with cancer, e.g., for reducing the risk of recurrence in subjects with cancer who undergo a surgical intervention, e.g., biopsy or resection. The methods can also be used to prepare a subject for a surgical procedure. Generally, the methods include administering a therapeutically effective amount of a COX-1 inhibitor (e.g., a COX-1 specific inhibitor that does not affect COX-2 at the dose administered), and/or other anti-platelet agents and/or TGF-β inhibition and/or IL-6 inhibition and/or inhibitors of the PD-1/PD-L1 axis, as described herein, to a subject who is in need of, or who has been determined to be in need of, such treatment.

In the present methods, the treatment is generally administered prior to surgical intervention, e.g., prior to biopsy or resection, e.g., 0-10 hours before, 0-8, 0-6, 0-4, 0-3, 0-2 hours before or preferably within a few (e.g., 30, 20, 15, 20, 10, or 5) minutes before surgery begins (i.e., prior to the first incision) or up to the half-life of the drug, and at least 0, 1, 2, 3, 4, 5, 6, 7, 8, 9, 10, 15, 20, 30, 45, 60, 90, or 120 minutes before the surgery begins. Thus, for example, in some embodiments a drug with a 4- to 6-hour half-life can be given a few minutes before surgery (e.g., by IV infusion) or up to 2-3 hours before the first incision, e.g., at about 2-3 hours before (“about” in the present sense indicates a variance of up to 10-20%). In some embodiments, only a single dose of the drug is given; in some embodiments, for very long surgical procedures, an additional dose may be given. In some embodiments, the drug is given long enough before the first incision to allow the drug to be distributed systemically. In some embodiments, the drug is administered locally, e.g., topically to the skin before the first incision, or immediately upon the first incision, e.g., with readministration during the surgical procedure.

As used herein, a surgical procedure includes any time a scalpel is used to make an incision, whether using cold instruments or lasers or cauterizing, that causes platelets to come to the area of the surgical site or wound, aggregate, and degranulate, in an amount sufficient to induce growth and proliferation of local or distant tumor cells, e.g., micrometastases. The surgical procedures can in some embodiments include major surgeries such as joint or organ replacement, laparotomy, or tumor resection, as well as less- or minimally-invasive surgeries (e.g., robotic, endoscopic); and needle biopsies for retrieving tissue samples, e.g., for biopsy/pathology. Typically, the surgical procedure includes incision beyond the epidermal layer, e.g., into or beyond subcutaneous layers (hypodermis) or dermis. In some embodiments, the use of a needle to obtain a liquid sample, e.g., a blood sample is not a surgical procedure.

As used in this context, to “treat” means to ameliorate at least one clinical parameter of the cancer. In some embodiments, the parameter is metastatic tumor size or growth rate (i.e., of distant metastatic disease or micrometastases), tumor recurrence, or metastasis, and an improvement would be a reduction in tumor size or no change in a normally fast growing tumor; a reduction or cessation of tumor growth; a reduction in, delayed, or no recurrence; or a reduction in, delayed, or no detectable metastasis. Administration of a therapeutically effective amount of a compound described herein for the treatment of a cancer would result in one or more of a reduction in tumor size or no change in a normally fast growing tumor; a reduction or cessation of tumor growth; or a reduction in, delayed, or no metastasis. In the present methods, e.g., a treatment designed to prevent recurrence of cancer, the treatment would be given before a tumor biopsy or before resection of a localized tumor (i.e., surgically removed). Without wishing to be bound by theory, such a treatment may work by keeping micrometastases dormant, e.g., by preventing them from being released from dormancy.

As used herein, the term “hyperproliferative” refer to cells having the capacity for autonomous growth, i.e., an abnormal state or condition characterized by rapidly proliferating cell growth. Hyperproliferative disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A “tumor” is an abnormal growth of hyperproliferative cells. “Cancer” refers to pathologic disease states, e.g., characterized by malignant tumor growth. The methods described herein can be used to treat cancer, e.g., solid tumors of epithelial origin, e.g., as defined by the ICD-O (International Classification of Diseases—Oncology) code (revision 3), section (8010-8790). Thus the methods can include treating subjects who are known or suspected of having solid tumors of epithelial origin. Cancers of epithelial origin can include pancreatic cancer (e.g., pancreatic adenocarcinoma), lung cancer (e.g., non-small cell lung carcinoma or small cell lung carcinoma), prostate cancer, breast cancer, renal cancer, ovarian cancer, or colon cancer. The methods can also be used to treat early preneoplastic cancers as a means to prevent the development of invasive cancer. In some embodiments, the methods are used to treat subjects who are suspected of having cancer, e.g., who are having a surgical biopsy (e.g., for diagnostic purposes), or who have been diagnosed with cancer and are having their cancer resected or surgically debulked.

“Micrometastases” as used herein are clusters of tumor cells that are up to about 0.2-2 mm³ and typically contain less than or equal to about 10⁶ viable tumor cells; as used herein, the term micrometastases also includes isolated tumor cell clusters (smaller than 0.2 mm³ or less than 200 cancer cells in one section). See, e.g., Schabel et al., Cancer. 1975 January; 35(1):15-24; Naidoo and Pinder, Surgeon. 2016 Aug. 3. pii: S1479-666X(16)30050-6; Hurst et al., Ther Adv Med Oncol. 2016 March; 8(2):126-37. They are associated with a number of cancers, including breast, colon, melanoma, prostate, pancreatic, cervical, thyroid, and other cancers, and typically appear in lung, kidney, and lymph node tissues.

COX-1 Specific Inhibitors

The present methods can include administration of a COX-1 inhibitor, preferably a COX-1 specific inhibitor (i.e., a COX-1 inhibitor that does not affect COX-2 at the dose administered) such as triflusal, and/or other anti-platelet agents. Thus the methods can include administering irreversible cyclooxygenase-1 inhibitors such as triflusal (2-acetyloxy-4-(trifluoromethyl)benzoic acid), aspirin (e.g., low dose aspirin), SC-560 (5-(4-Chlorophenyl)-1-(4-methoxyphenyl)-3-trifluoromethyl pyrazole), FR122047, mofezolac, P6, tenidap, ketorolac, Valeryl salicylate, and TFAP (Perrone et al., Curr Med Chem. 2010; 17(32):3769-805); Adenosine diphosphate (ADP) receptor inhibitors such as Clopidogrel (Plavix), Prasugrel (Effient), Ticagrelor (Brilinta), or Ticlopidine (Ticlid); phosphodiesterase inhibitors such as Cilostazol (Pletal); protease-activated receptor-1 (PAR-1) antagonists such as Vorapaxar (Zontivity); Glycoprotein IIB/IIIA (GPIIB/IIA) inhibitors such as Abciximab (ReoPro), Eptifibatide (Integrilin), or Tirofiban (Aggrastat); adenosine reuptake inhibitors such as dipyridamole (Persantine); and thromboxane inhibitors including thromboxane synthase inhibitors and thromboxane receptor antagonists such as terutroban. In some embodiments, the COX-1 inhibitor does not affect COX-2 activity at any dose.

TGF-β Inhibitors

The present methods can include administration of TGF-beta neutralizing therapies, including anti-TGF-beta antibodies (e.g. fresolimumab, Infliximab, Metelimumab, Lerdelimumab, GC-1008), antisense oligodeoxynucleotides (e.g., Trabedersen), and small molecule inhibitors of TGF-beta (e.g. LY2157299, LY2382770, Lucanix, Disitertide), (Wojtowicz-Praga, Invest New Drugs. 21(1): 21-32 (2003)), as well as TGF-beta receptor inhibitors such as Galunisertib, TEW-7197, PF-03446962, and IMC-TR1. See, e.g., Neuzillet et al., Pharmacology & Therapeutics 147:22-31 (2015);

IL-6 Inhibitors

The present methods can include administration of IL-6 inhibitors including anti-IL-6 antibodies (e.g. siltuximab, tocilizumab (atlizumab), sarilumab, olokizumab (CDP6038), elsilimomab, Clazakizumab, sirukumab (CNTO 136), ALX-0061 (nanobody) (Guo, et al., Cancer Treat Rev. 38(7):904-910 (2012)), or small molecule or peptide inhibitors, e.g., CPSI-2364, Semapimod, ARGX-109, FE301, FM101, or soluble gp130 or sgf130Fc (Scheller et al., Semin Immunol. 2014 February; 26(1):2-12).

Inhibitors of the PD-I/PD-L Axis

The present methods can include administration of an inhibitor of the Programmed cell death protein 1 (also known as PD-1 and CD279 (cluster of differentiation 279)/programmed death ligand-1 (PD-L1) axis, e.g., anti-PD1 (e.g., Nivolumab, pembrolizumab/MK-3475, Pidilizumab (CT-011), AMP-224, AMP-554), or anti-PDL1 (e.g., BMS-936559, MEDI4736, Atezolizumab (MPDL3280A), Avelumab (MSB0010718C)), see, e.g., Dolan and Gupta, Cancer Control. 2014 July; 21(3):231-7.

Pharmaceutical Compositions and Methods of Administration

The methods described herein include the use of pharmaceutical compositions comprising COX-1 selective inhibitors, other anti-platelet agents and/or TGF-β inhibition and/or IL-6 inhibition and/or inhibitors of the PD-1/PD-L1 axis, as well as combinations thereof, as active ingredients.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. In some embodiments, e.g., wherein the agent is administered locally, the composition can include epinephrine, e.g., to induce vasoconstriction to slow drug distribution relative to the site of injection or incision. The epinephrine can also be administered separately, and one or both can be administered by injection at or near the incision site, or into the wound or surgical site, e.g., using a gel. See, e.g., Tanaka et al., Anesth Prog. 2016 Spring; 63(1): 17-24; Gessler et al., Laryngoscope. 2001 October; 111(10): 1687-90; Smith et al., Cancer Chemother Pharmacol. 1999; 44(4):267-74.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In some embodiments, e.g., for local administration, liposomes (e.g., as described in Chahar and Cummings, J Pain Res. 2012; 5: 257-264, and U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

With regard to the dosage to be used, an “effective amount” is an amount sufficient to effect beneficial or desired results. For example, a therapeutic amount is one that achieves the desired therapeutic effect of reducing risk of cancer or cancer recurrence. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, the dose of Ketorolac is 20-30 or 20-50 mg/day for an approximately 70 kg human.

### EXAMPLES

The invention is further described in the following examples, which do not limit the scope of the invention described in the claims.

### Example 1. Pre-Operative Selective COX-1 Inhibition Inhibits Surgery-Induced Primary Tumor Growth and Metastasis

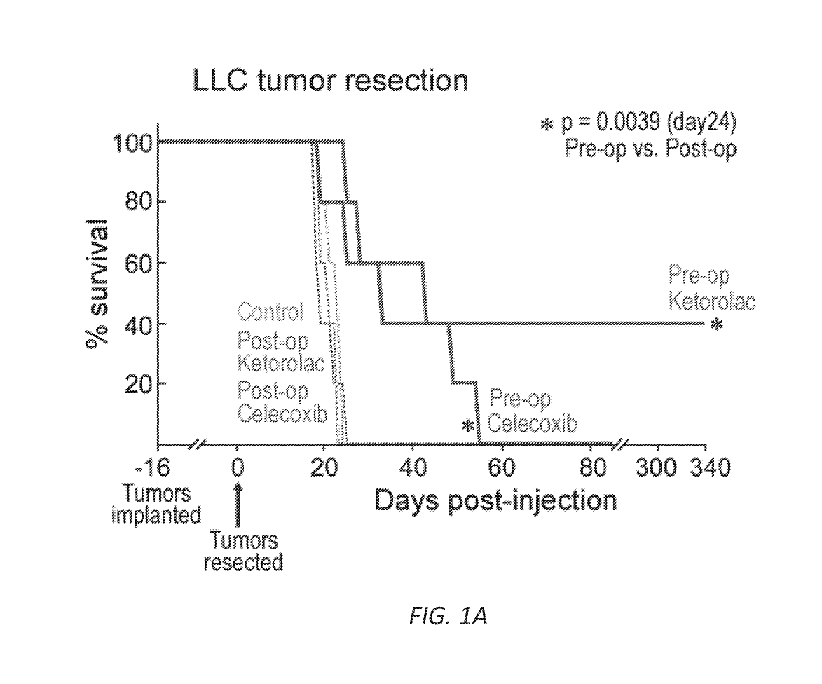

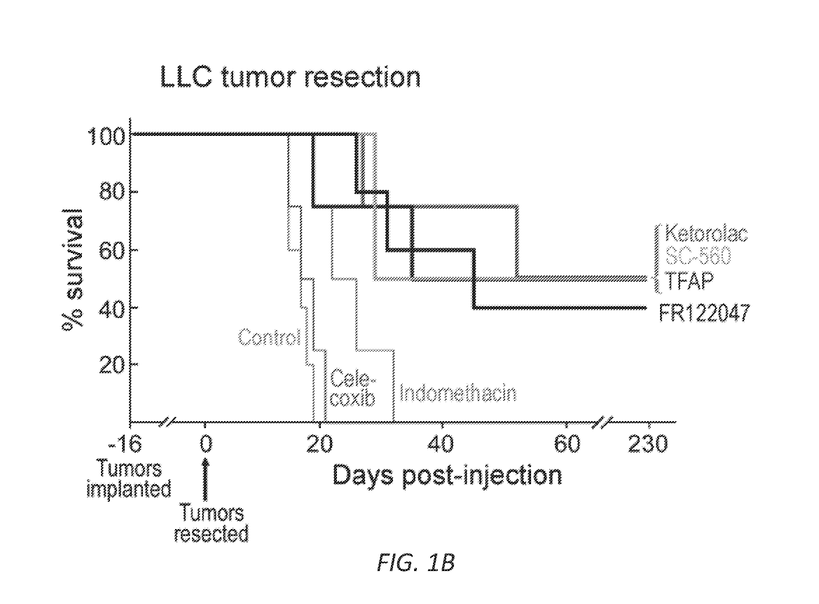

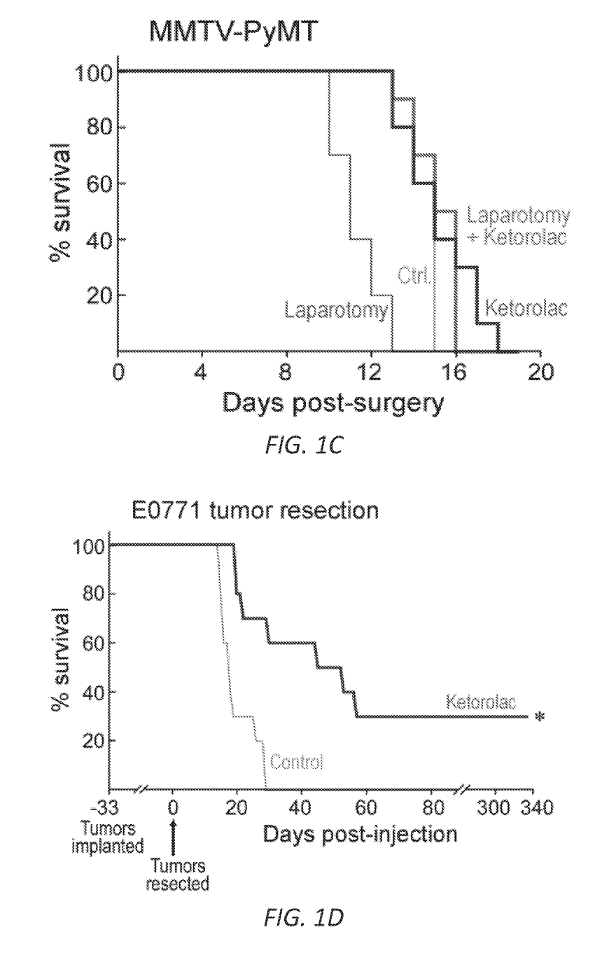

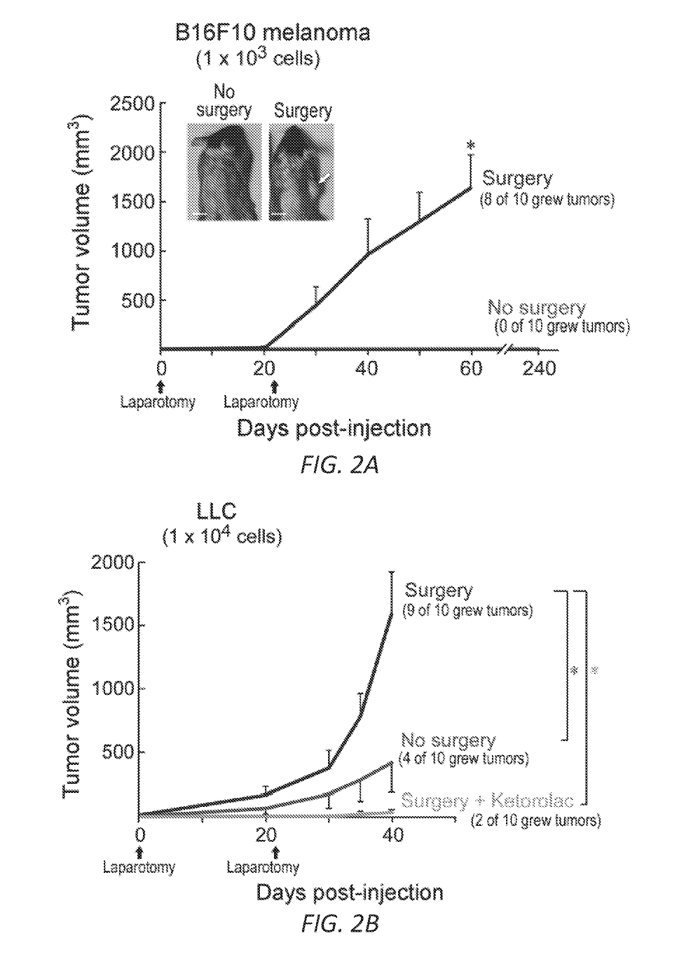

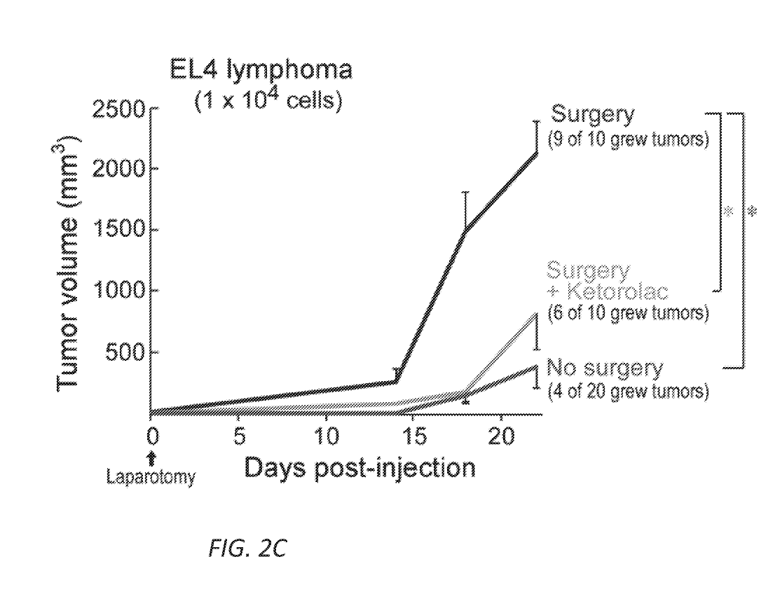

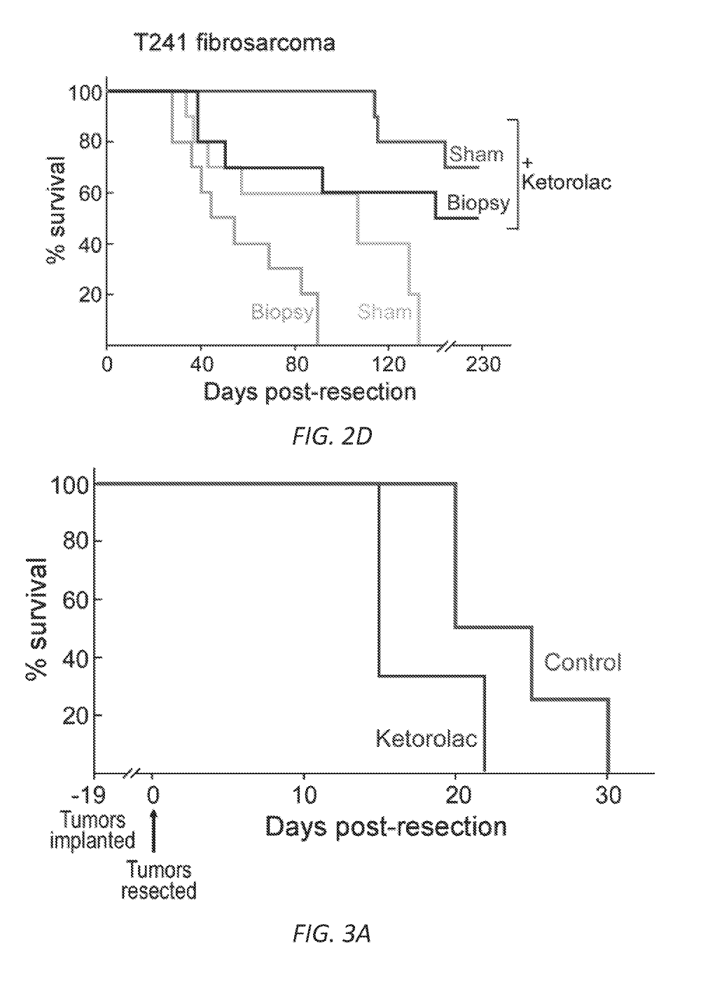

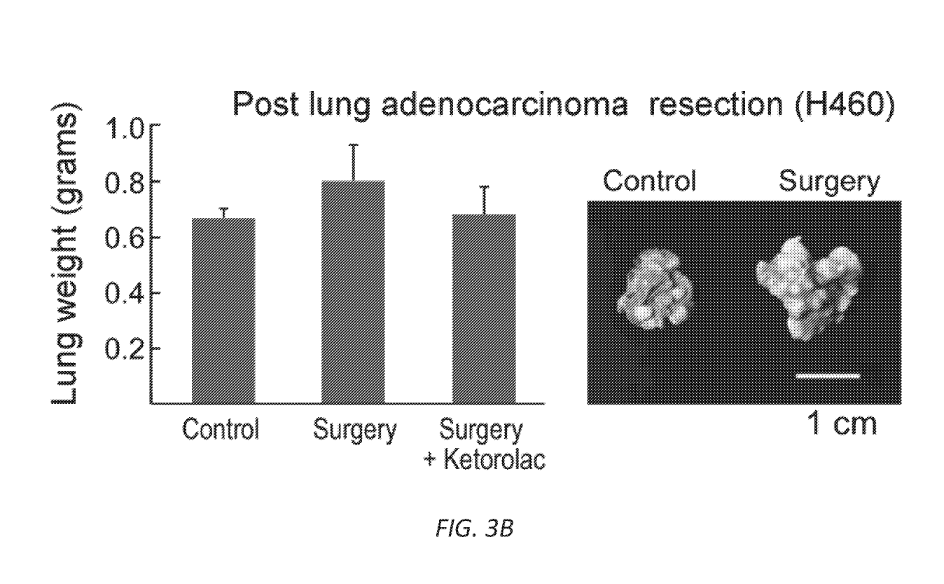

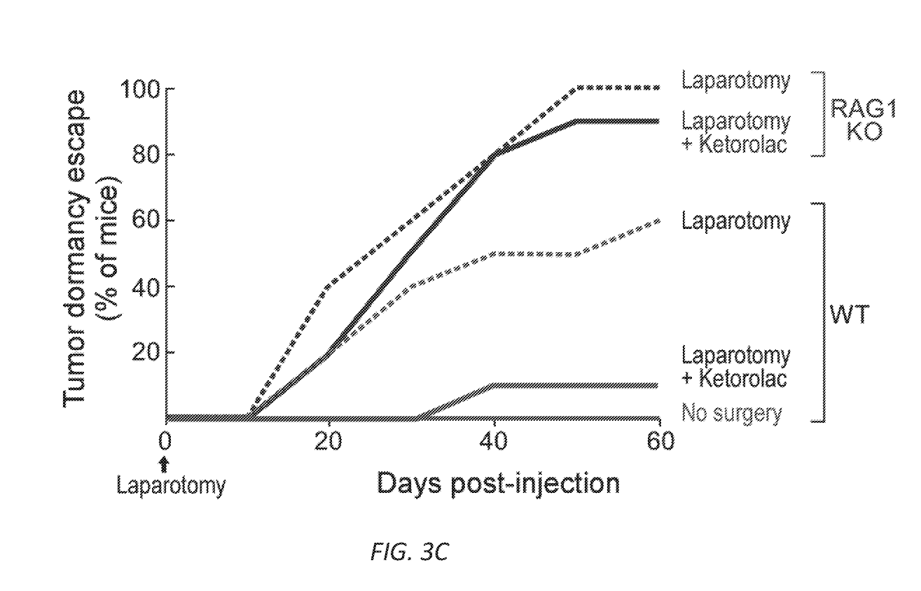

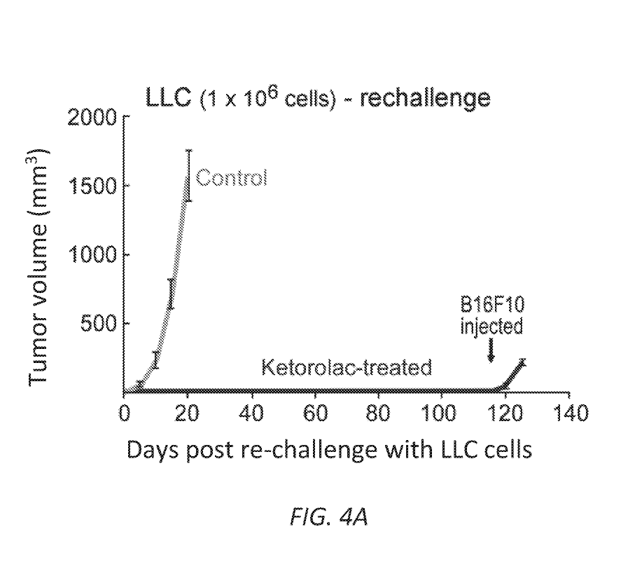

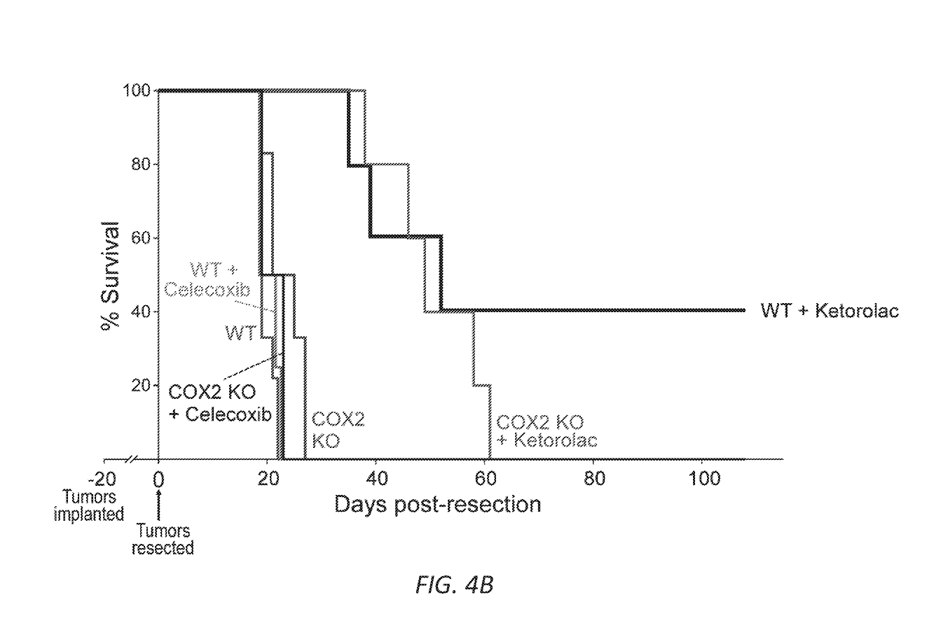

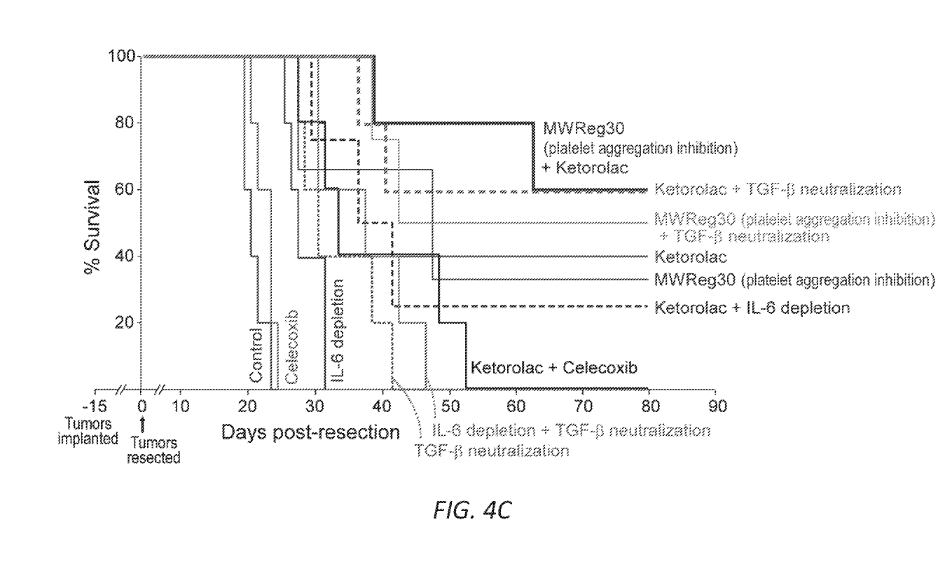

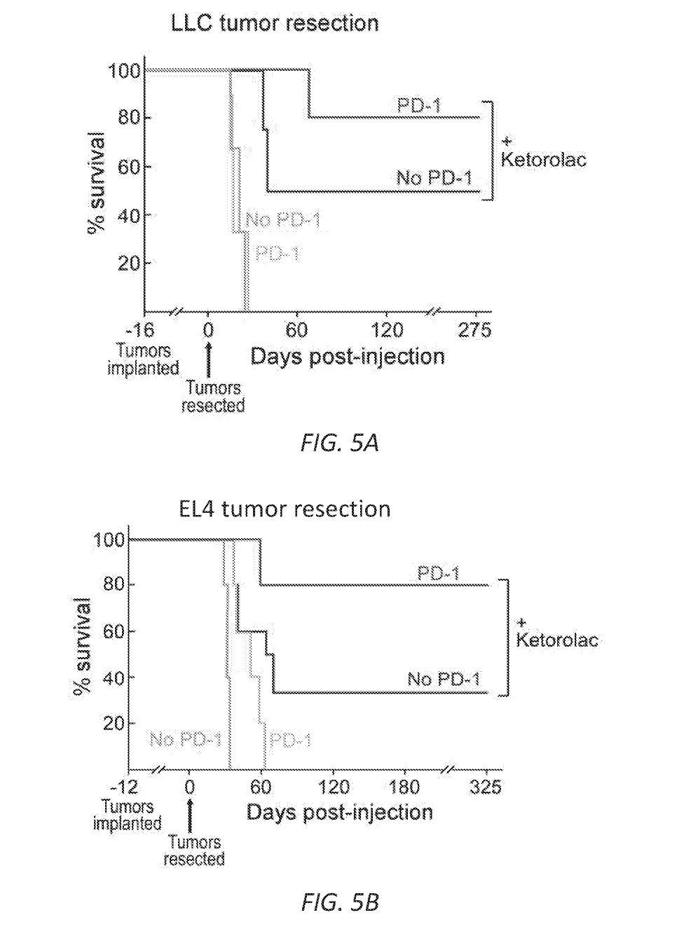

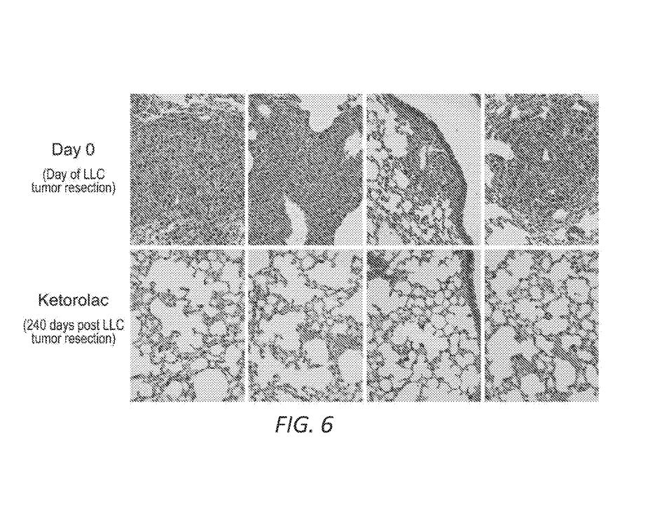

Experiments were performed to determine the effects of preoperative and post-operative Ketorolac (a COX-1/COX-2 inhibitor administered 7.5 mg/kg intraperitoneally (I.P.)) and celecoxib (a COX-2 inhibitor that does not inhibit COX-1 at therapeutic concentrations, administered 30 mg/kg bid po) survival in a spontaneous Lewis Lung carcinoma (LLC) lung metastasis model (see, e.g., O'Reilly et al., Cell. 1994 Oct. 21; 79(2):315-28; Panigraphy et al., J Clin Invest. 2002 October; 110(7):923-32; and Panigraphy et al., J Clin Invest. 2012 January; 122(1):178-91) after primary tumor resection. As shown in FIG. 1A, pre-operative, but not post-operative, ketorolac and celecoxib, prolonged survival. Pre-operative, but not post-operative ketorolac “cured” (provided long term survival in) approximately 50% of mice in a syngeneic LLC lung cancer model (see, e.g., O'Reilly et al., Cell. 1994 Oct. 21; 79(2):315-28). Immunohistochemistry confirmed the presence of micrometastasis on day of tumor resection with no evidence of tumor micrometastasis in mice treated with ketorolac treated for 180 to 200 days.

To determine whether the effects of Ketorolac were due to action on COX-1, the effects of COX-1 selective inhibitors (FR122047 (20 mg/kg i.p.); TFAP (30 mg/kg i.p.); and SC-560 (30 mg/kg i.p.)), a COX-2 selective inhibitor celecoxib (60 mg/kg/oral gavage), and a non-selective COX inhibitor indomethacin (3 mg/kg/i.p.); were evaluated. Doses were selected based on previous publications by the present inventors and others, and/or by analogy to the human dose. As shown in FIG. 1B, FR122047, TFAP, and SC-560 mimicked the action of ketorolac (7.5 mg/kg i.p.) in prolonging survival after primary LLC tumor resection.

This effect was not limited to lung cancer; as shown in FIGS. 1C-1D, in a surgery-stimulated spontaneous tumor model (a genetically engineered murine breast tumor model that develops spontaneous tumors after surgery, in this case laparotomy; MMTV-PyMT, see Guy et al., Mol Cell Biol. 1992, 12: 954-961; Fantozzi and Christofori, Breast Cancer Research20068:212), Ketorolac prolonged survival (FIG. 1C), and pre-operative ketorolac also prolonged survival in a spontaneous E0771 breast cancer metastasis model after primary tumor resection (Ewens et al., Anticancer Res. 2005 November-December; 25(6B):3905-15; FIG. 1D).

These results show that pre-operative selective cox-1 inhibition inhibits surgery-induced primary tumor growth and metastasis.

### Example 2. Pre-Operative Ketorolac Suppresses Surgery-Stimulated Primary Tumor Growth and Biopsy-Stimulated Metastasis

Surgery (i.e., laparotomy, which is a surgical procedure involving a large incision through the abdominal wall to gain access into the abdominal cavity, also known as a celiotomy) stimulated primary tumor dormancy escape in a B16F10 melanoma (1×10³ cells, see Kalpainen et al., PLoS One. 2007 Feb. 28; 2(2):e260 and Panigraphy et al., J Clin Invest. 2012 January; 122(1):178-91; FIG. 2A), Lewis lung carcinoma (LLC 1×10⁴ cells, FIG. 2B), and EL4 lymphoma (1×10⁴ cells, FIG. 2C). Ketorolac administration pre-surgically inhibited surgery-induced tumor dormancy escape (see FIGS. 2B-C).

In addition, liver biopsy alone stimulated lung metastasis post primary tumor resection in a T241 fibrosarcoma model (O'Reilly et al., Cell. 1994 Oct. 21; 79(2):315-28 and Panigraphy et al., J Clin Invest. 2012 January; 122(1):178-91). As shown in FIG. 2D, Ketorolac administration pre-surgically inhibited this biopsy-stimulated lung metastasis.

These results showed that pre-operative ketorolac suppresses surgery-stimulated primary tumor growth and biopsy-stimulated metastasis.

### Example 3. Adaptive Immunity is Critical to Anti-Metastatic Activity of Ketorolac

To determine whether the effects of Ketorolac were due to actions on the immune system, the effects of ketorolac administered prior to LLC tumor resection was determined in nude athymic C57BL/6 mice, SCID mice, and Rag1 KO Mice. As shown in FIG. 3AC, Ketorolac did not have anti-metastatic activity in nude athymic C57BL/6 mice (FIG. 3A), did not inhibit metastases in SCID mice after human lung tumor (H460) primary tumor resection (FIG. 3B), and did not inhibit laparotomy-induced tumor dormancy escape in Rag1 KO Mice (B16F10, 1×10³, FIG. 3C).

These results show that adaptive immunity is critical to the anti-metastatic activity of ketorolac.

### Example 4. Platelet Depletion/Anti-Aggregation Agent and TGFβ Inhibition Synergize with Ketorolac to Prevent Tumor Recurrence

To determine whether perioperative COX-1 inhibition produces a durable immune response to the cancer, immunocompetent mice (C57BL6) at 200 days after undergoing LLC tumor resection during which they were administered ketorolac preoperatively, were given a second tumor challenge with the same tumor types (LLC). As shown in FIG. 4A, the mice resisted a second tumor challenge with the same strain (LLC). In contrast, these same mice exhibited rapid tumor growth when later injected with a third tumor challenge with a different tumor-type (B16F10 melanoma), indicating tumor-specific immunity.

As shown in FIG. 4B, COX-1 inhibition with Ketorolac also prevented tumor metastasis when administered prior to surgery for LLC tumor resection in WT and COX-2 knockout (KO) mice, providing further evidence that COX-2 is not relevant for the anti-tumoral actions of ketorolac. In contrast, celecoxib did not prevent metastasis in WT and COX-2 KO mice.

To determine possible synergies with other agents that inhibit platelet aggregation or degranulation, or inhibit the downstream effects of platelet aggregation and/or degranulation, a COX-1 inhibitor was administered in combination with platelet depletion or aggregation inhibiting agent (MWReg30; 2 ug/mouse), a TGF-β inhibitor (1 mg/mouse), an IL-6 neutralizing antibody (1 mg/mouse), and a TGF-B inhibitor in combinations with MWReg30, alone or in combination with the COX-1 inhibitor ketorolac. As shown in FIG. 4C, each of the interventions prevented tumor metastasis post primary LLC tumor resection to some extent, with a combination of an anti-platelet agent (ant-CD41 antibody) and ketorolac, or a combination of TGF-B inhibitor in with an anti-platelet agent, being the most effective.

Thus in this surgery-stimulated (LLC tumor resection) model, inhibiting platelet aggregation or inhibiting inflammation (e.g., via IL-6 or TGF-beta neutralizing antibody) prevented tumor recurrence. This suggests that platelets and/or inflammation have a key role in surgery-stimulated tumor growth at the time of surgery in the perioperative time period.

### Example 5. Combination of Preoperative Ketorolac and Checkpoint Inhibitor Exhibit Synergistic Anti-Metastatic Activity

To determine whether checkpoint inhibitors, which help to maintain or enhance the T-cell mediated adaptive immune response, would prolong survival when administered preoperatively in combination with Ketorolac, experiments were performed as described above. As shown in FIGS. 5A-B, ketorolac plus anti-PD1 in a post-primary tumor resection model (where the primary tumor was LLC (5A) or EL4 (5B)) significantly increased long term survival compared to ketorolac or PD-1 alone.

These results showed that a combination of a preoperative COX-1 inhibitor and checkpoint inhibitor exhibit synergistic anti-metastatic activity.

### Example 6. Preoperative COX-1 Inhibition Triggers Clearance of Micrometastases

Hematoxylin & Eosin analysis of lungs at the time of surgery (LLC tumor resection) (Day 0) revealed abundant LLC tumor cells. In dramatic contrast, in lungs from mice treated 2 hours prior to surgery with i.p. ketorolac 7.5 mg/kg, no tumor cells were detectable after 240 days post-surgery (LLC tumor resection).

These results show that preoperative cox-1 inhibition triggers clearance of micrometastases and/or prevented growth or proliferation of dormant tumor cells, likely by an immune-mediated mechanism.

### REFERENCES

1. Shakhar, G. and S. Ben-Eliyahu, Potential prophylactic measures against postoperative immunosuppression: could they reduce recurrence rates in oncological patients? Ann Surg Oncol, 2003. 10(8): p. 972-92.
2. Fisher, B. and E. R. Fisher, Experimental evidence in support of the dormant tumor cell. Science, 1959. 130(3380): p. 918-9.
3. Avraham, R., et al., Synergism between immunostimulation and prevention of surgery-induced immune suppression: an approach to reduce post-operative tumor progression. Brain Behav Immun, 2010. 24(6): p. 952-8.
4. Benish, M. and S. Ben-Eliyahu, Surgery as a double-edged sword: a clinically feasible approach to overcome the metastasis-promoting effects of surgery by blunting stress and prostaglandin responses. Cancers (Basel), 2010. 2(4): p. 1929-51.
5. Brinton, L. A., et al., Cancer risk at sites other than the breast following augmentation mammoplasty. Ann Epidemiol, 2001. 11(4): p. 248-56.
6. Hedley, B. D. and A. F. Chambers, Tumor dormancy and metastasis. Adv Cancer Res, 2009. 102: p. 67-101.
7. Harach, H. R., K. O. Franssila, and V. M. Wasenius, Occult papillary carcinoma of the thyroid. A “normal” finding in Finland. A systematic autopsy study. Cancer, 1985. 56(3): p. 531-8.
8. Naumov, G. N., L. A. Akslen, and J. Folkman, Role of angiogenesis in human tumor dormancy: animal models of the angiogenic switch. Cell Cycle, 2006. 5(16): p. 1779-87.
9. Mathenge, E. G., et al., Core needle biopsy of breast cancer tumors increases distant metastases in a mouse model. Neoplasia, 2014. 16(11): p. 950-60.
10. Cuzick, J., et al., Preventive therapy for breast cancer: a consensus statement. Lancet Oncol, 2011. 12(5): p. 496-503.
11. Janne, P. A. and R. J. Mayer, Chemoprevention of colorectal cancer. N Engl J Med, 2000. 342(26): p. 1960-8.
12. Bresalier, R. S., et al., Cardiovascular events associated with rofecoxib in a colorectal adenoma chemoprevention trial. N Engl J Med, 2005. 352(11): p. 1092-102.
13. McGettigan, P. and D. Henry, Cardiovascular risk and inhibition of cyclooxygenase: a systematic review of the observational studies of selective and nonselective inhibitors of cyclooxygenase 2. JAMA, 2006. 296(13): p. 1633-44.
14. Warner, T. D., et al., Nonsteroid drug selectivities for cyclo-oxygenase-1 rather than cyclo-oxygenase-2 are associated with human gastrointestinal toxicity: a full in vitro analysis. Proc Natl Acad Sci USA, 1999. 96(13): p. 7563-8.
15. Gillis, J. C. and R. N. Brogden, Ketorolac. A reappraisal of its pharmacodynamic and pharmacokinetic properties and therapeutic use in pain management. Drugs, 1997. 53(1): p. 139-88.
16. Diblasio, C. J., et al., Ketorolac: safe and effective analgesia for the management of renal cortical tumors with partial nephrectomy. J Urol, 2004. 171(3): p. 1062-5.
17. Colacchio, T. A., M. P. Yeager, and L. W. Hildebrandt, Perioperative immunomodulation in cancer surgery. Am J Surg, 1994. 167(1): p. 174-9.
18. Shapiro, J., et al., Anesthetic drugs accelerate the progression of postoperative metastases of mouse tumors. J Clin Invest, 1981. 68(3): p. 678-85.
19. Melamed, R., et al., Suppression of natural killer cell activity and promotion of tumor metastasis by ketamine, thiopental, and halothane, but not by propofol: mediating mechanisms and prophylactic measures. Anesth Analg, 2003. 97(5): p. 1331-9.
20. Beilin, B., et al., The effects of postoperative pain management on immune response to surgery. Anesth Analg, 2003. 97(3): p. 822-7.
21. Markovic, S. N., P. R. Knight, and D. M. Murasko, Inhibition of interferon stimulation of natural killer cell activity in mice anesthetized with halothane or isoflurane. Anesthesiology, 1993. 78(4): p. 700-6.
22. Forget, P., et al., Neutrophil:lymphocyte ratio and intraoperative use of ketorolac or diclofenac are prognostic factors in different cohorts of patients undergoing breast, lung, and kidney cancer surgery. Ann Surg Oncol, 2013. 20 Suppl 3: p. S650-60.
23. Demicheli, R., et al., Breast cancer recurrence dynamics following adjuvant CMF is consistent with tumor dormancy and mastectomy-driven acceleration of the metastatic process. Ann Oncol, 2005. 16(9): p. 1449-57.
24. Pascual, M., et al., Randomized clinical trial comparing inflammatory and angiogenic response after open versus laparoscopic curative resection for colonic cancer. Br J Surg, 2011. 98(1): p. 50-9.
25. Abramovitch, R., et al., Stimulation of tumour growth by wound-derived growth factors. Br J Cancer, 1999. 79(9-10): p. 1392-8.
26. Buvanendran, A. and J. S. Kroin, Multimodal analgesia for controlling acute postoperative pain. Curr Opin Anaesthesiol, 2009. 22(5): p. 588-93.
27. Farooqui, M., et al., COX-2 inhibitor celecoxib prevents chronic morphine-induced promotion of angiogenesis, tumour growth, metastasis and mortality, without compromising analgesia. Br J Cancer, 2007. 97(11): p. 1523-31.
28. Glasner, A., et al., Improving survival rates in two models of spontaneous postoperative metastasis in mice by combined administration of a beta-adrenergic antagonist and a cyclooxygenase-2 inhibitor. J Immunol, 2010. 184(5): p. 2449-57.
29. Gupta, K., et al., Morphine stimulates angiogenesis by activating proangiogenic and survival-promoting signaling and promotes breast tumor growth. Cancer Res, 2002. 62(15): p. 4491-8.
30. Bovill, J. G., Surgery for cancer: does anesthesia matter? Anesth Analg, 2010. 110(6): p. 1524-6.
31. Backhus, L. M., et al., Perioperative cyclooxygenase 2 inhibition to reduce tumor cell adhesion and metastatic potential of circulating tumor cells in non-small cell lung cancer. J Thorac Cardiovasc Surg, 2006. 132(2): p. 297-303.
32. Wang, Z. Y., et al., Which has the least immunity depression during postoperative analgesia-morphine, tramadol, or tramadol with lomoxicam? Clin Chim Acta, 2006. 369(1): p. 40-5.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing the risk of cancer occurrence or recurrence in a subject undergoing a surgical procedure, the method comprising administering to the subject a therapeutically effective amount of one or more of:
(i) one or more anti-platelet agents, preferably a COX-1 specific inhibitor and/or anti-CD41 antibody,
(ii) a TGF-β inhibitor;
(iii) an IL-6 inhibitor; and/or
(iv) a checkpoint inhibitor, preferably a Programmed cell death protein 1 (PD-1) or programmed death ligand-1 (PD-L1) inhibitor,
wherein the administering is performed before the surgical procedure.

2. A method of inducing an adaptive immune response to a cancer in a subject who has cancer, the method comprising administering to the subject a therapeutically effective amount of one or more of:
(i) an anti-platelet agent, preferably a COX-1 specific inhibitor,
(ii) TGF-β inhibitor;
(iii) an IL-6 inhibitor; and/or
(iv) a Programmed cell death protein 1 (PD-1) or programmed death ligand-1 (PD-L1) inhibitor,
and subsequently performing a surgical procedure in the subject.

3. The method of claim 1, wherein the anti-platelet agent is a COX-1 Specific Inhibitor.

4. The method of claim 3, wherein the COX-1 Specific Inhibitor is triflusal (2-acetyloxy-4-(trifluoromethyl)benzoic acid), aspirin, SC-560 (5-(4-Chlorophenyl)-1-(4-methoxyphenyl)-3-trifluoromethyl pyrazole), FR122047, mofezolac, P6, tenidap, ketorolac, Valeryl salicylate, or TFAP.

5. The method of claim 1, wherein the anti-platelet agent is an adenosine diphosphate (ADP) receptor inhibitor, a phosphodiesterase inhibitor; a protease-activated receptor-1 (PAR-1) antagonist; a Glycoprotein IIB/IIIA (GPIIB/IIA) inhibitor; an anti-CD41 antibody; an adenosine reuptake inhibitor; or a thromboxane inhibitor.

6. The method of claim 3, wherein the COX-1 inhibitor does not affect COX-2 activity at the administered dose, or at any dose.

7. The method of claim 1, wherein the TGF-β inhibitor is an anti-TGF-beta antibody, antisense oligodeoxynucleotide, small molecule inhibitor of TGF-beta, or TGF-beta receptor inhibitor.

8. The method of claim 7, wherein the anti-TGF-beta antibody is fresolimumab, Infliximab, Metelimumab, Lerdelimumab, GC-1008); the antisense oligodeoxynucleotide is Trabedersen; the small molecule inhibitor of TGF-beta is LY2157299, LY2382770, Lucanix, or Disitertide; or the TGF-beta receptor inhibitor is Galunisertib, TEW-7197, PF-03446962, or IMC-TR1.

9. The method of claim 1, wherein the IL-6 Inhibitor is an anti-IL-6 antibody or small molecule or peptide inhibitor.

10. The method of claim 9, wherein the anti-IL-6 antibody is siltuximab, tocilizumab (atlizumab), sarilumab, olokizumab (CDP6038), elsilimomab, Clazakizumab, sirukumab (CNTO 136), or ALX-0061; or the small molecule or peptide inhibitor is CPSI-2364, Semapimod, ARGX-109, FE301, FM101, or soluble gp130 or sgf130Fc.

11. The method of claim 1, wherein the inhibitor of PD-1/PD-L1 is an anti-PD 1 or anti-PD-L1 antibody.

12. The method of claim 11, wherein the anti-PD 1 is Nivolumab, pembrolizumab/MK-3475, Pidilizumab (CT-011), AMP-224, or AMP-554; or the anti-PDL1 is BMS-936559, MEDI4736, Atezolizumab (MPDL3280A), or Avelumab (MSB0010718C).

13. The method of claim 1, wherein the administering is performed within 4 hours before the surgical procedure begins, or within 30 hours before the surgical procedure begins.

14. (canceled)

15. The method of claim 1, wherein the surgical procedure is a biopsy or surgical resection of a tumor.

16. The method of claim 1, wherein the administering is systemic or local to the surgical site.

17. (canceled)

18. The method of claim 16, wherein the administering is local to the surgical site, further comprising administering epinephrine to the surgical site.

19. The method of claim 1, wherein the subject has been diagnosed with a cancer.

20. The method of claim 19, wherein the surgical procedure is cancer resection.

21. (canceled)

22. The method of claim 2, wherein the surgical procedure is unrelated to the cancer.

23.-44. (canceled)

45. A method of preparing a subject for a surgical procedure, the method comprising administering to the subject a therapeutically effective amount of one or more of:
(i) an anti-platelet agent, preferably a COX-1 specific inhibitor,
(ii) TGF-β inhibitor;
(iii) an IL-6 inhibitor; and/or
(iv) a Programmed cell death protein 1 (PD-1) or programmed death ligand-1 (PD-L1) inhibitor,
and subsequently performing a surgical procedure in the subject.

46. (canceled)
